(19) United States
(12) Patent Application Publication
(10) Pub. No.: US 2024/0172983 A1
(43) Pub. Date: May 30, 2024

(54) STRETCHABLE DEVICE

(71) Applicant: Murata Manufacturing Co., Ltd., Nagaokakyo-shi (JP)

(72) Inventor: Nobuyasu HORI, Nagaokakyo-shi (JP)

(21) Appl. No.: 18/434,958

(22) Filed: Feb. 7, 2024

Related U.S. Application Data
(63) Continuation of application No. PCT/JP2022/037797, filed on Oct. 11, 2022.

(30) Foreign Application Priority Data
Nov. 11, 2021 (JP) 2021-184266

(51) Int. Cl. A61B 5/271; A61B 5/256; A61B 5/257

(52) U.S. Cl. CPC A61B 5/271; A61B 5/256; A61B 5/257

(57) ABSTRACT

A stretchable device that includes: a stretchable substrate that includes: a main body portion having a first main surface and a second main surface facing the first main surface, and a projecting piece portion connected to the main body portion, the projecting piece portion having a third main surface continuous with the first main surface and a fourth main surface continuous with the second main surface and facing the third main surface; a stretchable wiring, wherein at least a part of the stretchable wiring is arranged on the first main surface of the main body portion of the stretchable substrate; and a bioelectrode arranged on the third main surface of the projecting piece portion of the stretchable substrate and electrically connected to the stretchable wiring.

### CROSS REFERENCE TO RELATED APPLICATIONS

The present application is a continuation of International application No. PCT/JP2022/037797, filed Oct. 11, 2022, which claims priority to Japanese Patent Application No. 2021-184266, filed Nov. 11, 2021, the entire contents of each of which are incorporated herein by reference.

### TECHNICAL FIELD

The present disclosure relates to a stretchable device.

### BACKGROUND ART

Conventionally, as a stretchable device, there is one described in Japanese Patent Application Laid-Open No. 2019-96826 (Patent Document 1). A stretchable device includes a stretchable substrate, a stretchable wiring formed on a first main surface of the stretchable substrate, and an electrode formed on the same first main surface as the stretchable wiring and connected to the stretchable wiring. Then, a side of the first main surface of the stretchable substrate is attached to a living body, and a biological signal is detected by the electrode.

### SUMMARY OF THE INVENTION

In the conventional stretchable device as described above, since the side of the first main surface of the stretchable substrate is attached to a living body, the stretchable wiring comes into contact with the living body. As described above, when the stretchable wiring comes into contact with a living body, there is a problem that noise occurs in a biological signal to be detected. Further, when the stretchable wiring comes into contact with skin, there is a problem of giving discomfort to a subject.

In view of the above, an object of the present disclosure is to provide a stretchable device capable of reducing noise of a biological signal and reducing discomfort of a subject.

In order to solve the above problem, a stretchable device according to an aspect of the present disclosure includes: a stretchable substrate that includes: a main body portion having a first main surface and a second main surface facing the first main surface, and a projecting piece portion connected to the main body portion, the projecting piece portion having a third main surface continuous with the first main surface and a fourth main surface continuous with the second main surface and facing the third main surface; a stretchable wiring, wherein at least a part of the stretchable wiring is arranged on the first main surface of the main body portion of the stretchable substrate; and a bioelectrode arranged on the third main surface of the projecting piece portion of the stretchable substrate and electrically connected to the stretchable wiring.

Here, “on the main surface” refers to not an absolute direction such as a vertically upward direction defined in the direction of gravity but a direction toward the outer side between the outer side and the inner side of a substrate having the main surface as a boundary. Therefore, “on the main surface” is a relative direction determined by a direction of the main surface. Further, “on” a certain element includes not only a position immediately above in contact with the element (on), but also an upper position away from the element, that is, an upper position with another object on the element interposed between them or an upper position with a space between them (above).

According to the above aspect, at least a part of the stretchable wiring is arranged on the first main surface, and the second main surface is a surface to be a side attached to a living body. For this reason, when the stretchable device is attached to a living body and a biological signal is detected, a side of the second main surface of the main body portion is attached to the living body, and at least a part of the stretchable wiring is located on the side opposite to the living body and hardly comes into contact with the living body.

Further, the bioelectrode is arranged on the third main surface of the projecting piece portion connected to the main body portion. For this reason, when the stretchable device is attached to a living body and a biological signal is detected, for example, the projecting piece portion is bent toward the side of the second main surface, and the bioelectrode is located on the second main surface side with respect to the main body portion, so that a biological signal can be detected as the bioelectrode is brought into contact with the living body.

Therefore, it is possible to reduce noise of a biological signal generated by the stretchable wiring coming into contact with a living body, and it is possible to reduce discomfort of a subject generated by the stretchable wiring coming into contact with skin.

Preferably, in an embodiment of the stretchable device, the main body portion extends in a first direction, and the projecting piece portion is connected to an end edge of the main body portion located at an end portion in a second direction orthogonal to the first direction of the main body portion.

Here, “extends in a first direction” refers to extending along one path rather than extending in a manner branched into a plurality of paths, and includes not only extending linearly but also extending in a curved, meandering, or spiral shape.

According to the above embodiment, the main body portion can be wound around a living body, and a biological signal can be detected in a wider range. As a result, detection accuracy can be enhanced.

Preferably, in an embodiment of the stretchable device, the projecting piece portion is bendable toward a side of the second main surface.

According to the above embodiment, since the projecting piece portion can be bent toward the side of the second main surface, when the stretchable device is used by bending the projecting piece portion toward the side of the second main surface, the bioelectrode is located on the side of the second main surface with respect to the main body portion. By the above, at least a part of the stretchable wiring hardly comes into contact with a living body when the bioelectrode is brought into contact with the living body and a biological signal is detected.

Preferably, in an embodiment of the stretchable device, the projecting piece portion is in a bent position toward a side of the second main surface.

According to the above embodiment, since the projecting piece portion is bent toward the side of the second main surface, the bioelectrode is located on the side of the second main surface with respect to the main body portion. For this reason, the bioelectrode is located on the side of the second main surface with respect to the main body portion. By the above, at least a part of the stretchable wiring hardly comes into contact with a living body when the bioelectrode is brought into contact with the living body and a biological signal is detected.

Preferably, in an embodiment of the stretchable device, the main body portion extends in a first direction, and a center of the bioelectrode is arranged at a central portion in a second direction orthogonal to the first direction of the main body portion.

Here, the central portion of the main body portion refers to a central region obtained by dividing the first main surface into three equal parts in the second direction.

According to the above embodiment, since the center of the bioelectrode is arranged at a central portion of the main body portion, it is easy to install the bioelectrode at a desired position of a living body when the main body portion is attached to the living body.

Preferably, in an embodiment of the stretchable device, the stretchable device further includes an adhesive layer on the second main surface.

According to the above embodiment, since the adhesive layer is provided on the second main surface, for example, the projecting piece portion can be fixed to the second main surface by the adhesive layer as the projecting piece portion is bent toward the side of the second main surface.

Preferably, in an embodiment of the stretchable device, the stretchable device further includes an adhesive layer on the second main surface, in which the projecting piece portion is fixed to the second main surface by the adhesive layer.

According to the above embodiment, since the projecting piece portion is fixed to the second main surface by the adhesive layer, it is possible to prevent positional displacement of the bioelectrode with respect to a living body. By the above, deviation of a signal detection position can be prevented, and detection accuracy can be enhanced.

Further, a stretchable device according to an aspect of the present disclosure includes: a stretchable substrate that is stretchable; a stretchable wiring that is provided on the stretchable substrate and is stretchable; and a bioelectrode that is provided on the stretchable substrate, is electrically connected to the stretchable wiring, and is to be in contact with a living body, in which the stretchable substrate includes: a main body portion that includes a first main surface and a second main surface facing the first main surface; and a projecting piece portion that is connected to the main body portion and includes a third main surface and a fourth main surface facing the third main surface, at least a part of the stretchable wiring is arranged on the first main surface, the bioelectrode is arranged on the fourth main surface, the projecting piece portion is arranged with respect to the main body portion such that the third main surface is along the first main surface and the fourth main surface is along the second main surface, and the second main surface is a surface to be a side attached to the living body.

According to the above aspect, at least a part of the stretchable wiring is arranged on the first main surface, and the second main surface is a surface to be a side attached to a living body. For this reason, when the stretchable device is attached to a living body and a biological signal is detected, a side of the second main surface of the main body portion is attached to the living body, and at least a part of the stretchable wiring is located on the side opposite to the living body and hardly comes into contact with the living body.

Further, the bioelectrode is arranged on the fourth main surface, and the projecting piece portion is arranged with respect to the main body portion such that the third main surface is along the first main surface and the fourth main surface is along the second main surface. For this reason, the bioelectrode is located on the side of the second main surface with respect to the main body portion. By the above, at least a part of the stretchable wiring hardly comes into contact with a living body when the bioelectrode is brought into contact with the living body and a biological signal is detected.

Therefore, it is possible to reduce noise of a biological signal generated by the stretchable wiring coming into contact with a living body, and it is possible to reduce discomfort of a subject generated by the stretchable wiring coming into contact with skin.

Preferably, in an embodiment of the stretchable device, the main body portion extends in a first direction, and includes: a plurality of individual piece portions that include the first main surface and the second main surface and are arranged in the first direction; and a coupling portion that includes a fifth main surface and a sixth main surface facing the fifth main surface and connects two of the individual piece portions adjacent to each other in the first direction, each of the individual piece portions and the coupling portion are arranged such that the first main surface and the sixth main surface face each other, the projecting piece portion is connected to the coupling portion, the stretchable wiring includes an individual piece side wiring portion arranged on the first main surface and a coupling side wiring portion arranged on the sixth main surface, and the individual piece side wiring portion and the coupling side wiring portion are electrically connected.

According to the above embodiment, a length in the first direction of the main body portion can be adjusted by adjusting the number of individual piece portions. Further, the main body portion can be manufactured separately, and the yield is improved.

Preferably, in an embodiment of the stretchable device, the main body portion extends in a first direction, and includes: a plurality of individual piece portions that include the first main surface and the second main surface and are arranged in the first direction; and a coupling portion that includes a fifth main surface and a sixth main surface facing the fifth main surface and connects two of the individual piece portions adjacent to each other in the first direction, each of the individual piece portions and the coupling portion are arranged such that the first main surface and the sixth main surface face each other, the projecting piece portion is connected to the individual piece portion, the stretchable wiring includes an individual piece side wiring portion arranged on the first main surface and a coupling side wiring portion arranged on the sixth main surface, and the individual piece side wiring portion and the coupling side wiring portion are electrically connected.

According to the above embodiment, a length in the first direction of the main body portion can be adjusted by adjusting the number of individual piece portions. Further, the main body portion can be manufactured separately, and the yield is improved.

Preferably, in an embodiment of the stretchable device, the main body portion extends in a first direction and includes a plurality of individual piece portions including the first main surface and the second main surface and arranged in the first direction, the projecting piece portion is connected to each of the individual piece portions, the stretchable wiring includes an individual piece side wiring portion arranged on the first main surface, between two of the individual piece portions adjacent to each other in the first direction, an end portion of a first of the individual piece portions on a side of a second of the individual piece portions and an end portion of the second of the individual piece portions on a side of the first of the individual piece portions are bent and coupled such that a first main surface of the end portion of the first of the individual piece portions and a first main surface of the end portion of the second of the individual piece portions face each other, and the individual piece side wiring portion arranged in the first of the individual piece portions and the individual piece side wiring portion arranged in the second of the individual piece portions are electrically connected.

According to the above embodiment, a length in the first direction of the main body portion can be adjusted by adjusting the number of individual piece portions. Further, the main body portion can be manufactured separately, and the yield is improved.

Preferably, in an embodiment of the stretchable device, the main body portion extends in a first direction and has a first end portion and a second end portion on both sides in the first direction, the stretchable device further including: a connection terminal connected to the first end portion and connectable to the second end portion.

According to the above embodiment, the stretchable device is easily fixed to a subject by having the connection terminal, and deviation of a signal detection position can be prevented.

Preferably, in an embodiment of the stretchable device, a plurality of the stretchable wirings, a plurality of the projecting piece portions, and a plurality of the bioelectrodes are provided, the main body portion extends in a first direction and has a first end portion and a second end portion on both sides in the first direction, each of the plurality of stretchable wirings extends along the first direction from one end portion of the first end portion and the second end portion, each of the plurality of bioelectrodes is electrically connected to the plurality of stretchable wirings, and in the plurality of stretchable wirings, as a distance in the first direction from the one end portion to a portion connected to the bioelectrode increases, a width of the stretchable wiring increases.

According to the above embodiment, a biological signal is weak and is likely to attenuate while being transmitted from a detection position to a detector. By increasing a width of the stretchable wiring according to a transmission distance, signal attenuation hardly occurs in long-distance transmission, and a signal can be detected with high accuracy regardless of a signal transmission distance.

Preferably, in an embodiment of the stretchable device, the main body portion extends in a first direction, and the stretchable substrate further includes a connection portion that is connected to an end edge located at an end portion in a second direction orthogonal to the first direction in the main body portion and in which an end portion of the stretchable wiring is arranged, the stretchable device further including: a connection terminal attached to the connection portion and connected to the end portion of the stretchable wiring.

According to the above embodiment, since the connection terminal is provided, for example, an external device can be connected to the connection terminal and signal data can be transmitted to the external device.

Preferably, in an embodiment of the stretchable device, a plurality of the stretchable wirings, a plurality of the projecting piece portions, and a plurality of the bioelectrodes are provided, each of the plurality of stretchable wirings extends from the connection terminal to the plurality of bioelectrodes, and in the plurality of stretchable wirings, as a distance in the first direction from a portion connected to the connection terminal to a portion connected to the bioelectrode increases, a width of the stretchable wiring increases.

According to the above embodiment, a biological signal is weak and is likely to attenuate while being transmitted from a detection position to a detector. By increasing a width of the stretchable wiring according to a transmission distance, signal attenuation hardly occurs in long-distance transmission, and a signal can be detected with high accuracy regardless of a signal transmission distance.

According to the stretchable device according to an aspect of the present disclosure, noise of a biological signal can be reduced, and discomfort of a subject can be reduced.

### BRIEF EXPLANATION OF THE DRAWINGS

FIG. 1 is a plan view illustrating a first embodiment of a stretchable device.

FIG. 2 is a sectional view taken along II-II of FIG. 1.

FIG. 3 is a plan view illustrating a preparation state for attachment of the stretchable device.

FIG. 4 is a sectional view taken along IV-IV of FIG. 3.

FIG. 5 is an explanatory view illustrating a use state of the stretchable device.

FIG. 6 is a plan view illustrating a second embodiment of the stretchable device.

FIG. 7 is a sectional view taken along VII-VII of FIG. 6.

FIG. 8 is a plan view illustrating a third embodiment of the stretchable device.

FIG. 9 is a sectional view taken along IX-IX of FIG. 8.

FIG. 10 is a plan view illustrating a fourth embodiment of the stretchable device.

FIG. 11 is a sectional view taken along XI-XI of FIG. 10.

FIG. 12 is a plan view illustrating a fifth embodiment of the stretchable device.

FIG. 13 is a sectional view taken along XIII-XIII of FIG. 12.

FIG. 14 is a plan view illustrating a sixth embodiment of the stretchable device.

FIG. 15 is a plan view illustrating a seventh embodiment of the stretchable device.

### DETAILED DESCRIPTION OF THE INVENTION

Hereinafter, a stretchable device according to an aspect of the present disclosure will be described in detail with reference to an illustrated embodiment. Note that the drawings include some schematic drawings, and may not reflect actual dimensions or ratios.

### First Embodiment

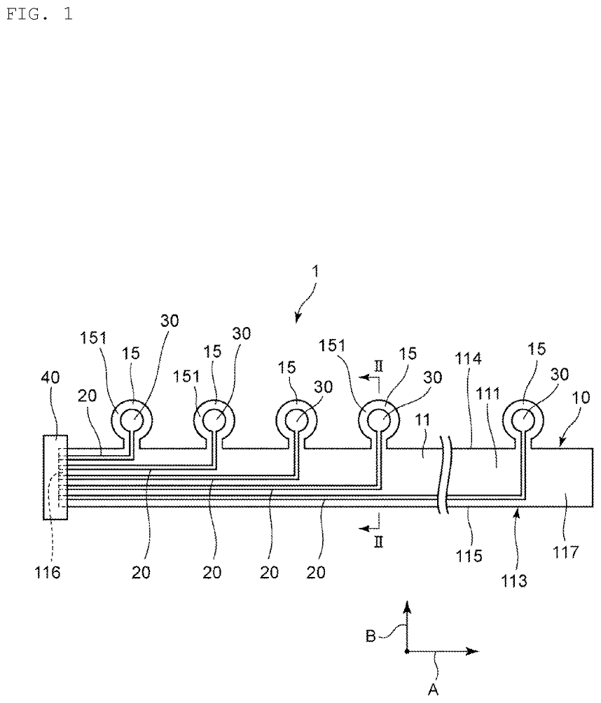

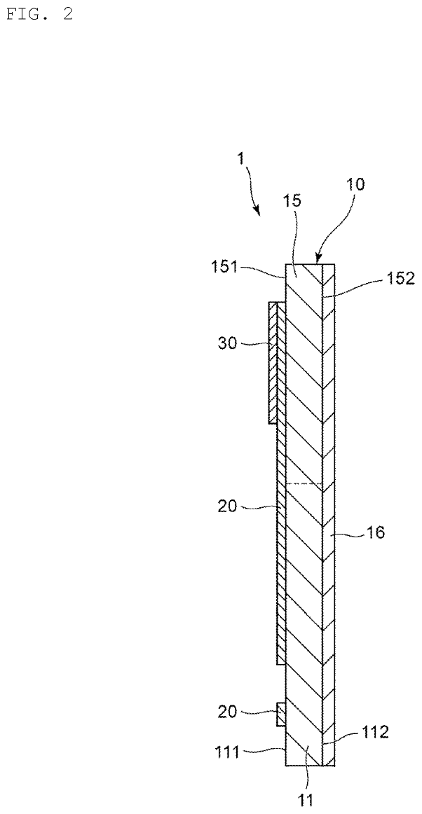

FIG. 1 is a plan view illustrating a first embodiment of a stretchable device. FIG. 2 is a sectional view taken along II-II of FIG. 1. The stretchable device is attached to a living body and used to measure a biological signal. Hereinafter, the side of the stretchable device attached to a living body is also referred to as a back side, and the side opposite to the back side is also referred to as a front side. The plan view of the stretchable device corresponds to a diagram of the front side of the stretchable device.

As illustrated in FIGS. 1 and 2, a stretchable device 1 includes a stretchable substrate 10 that is stretchable, a stretchable wiring 20 that is provided on the stretchable substrate 10 and is stretchable, and a bioelectrode 30 that is provided on the stretchable substrate 10, is electrically connected to the stretchable wiring 20, and is to be in contact with a living body.

The stretchable substrate 10 includes a sheet-like main body portion 11 and a plurality of sheet-like projecting piece portions 15. The main body portion 11 includes a resin material having elasticity, for example, thermoplastic polyurethane (TPU). Similarly to the main body portion 11, the projecting piece portion 15 includes a resin material having elasticity, for example, thermoplastic polyurethane (TPU). A material forming the main body portion 11 and a material forming the projecting piece portion 15 may be the same or different. A thickness of the main body portion 11 is, for example, 20 μm to 200 μm. A thickness of the projecting piece portion 15 is, for example, 1 μm to 100 μm. A thickness of the main body portion 11 and a thickness of the projecting piece portion 15 may be the same or different.

The main body portion 11 includes a first main surface 111, a second main surface 112 facing the first main surface 111, and a peripheral edge 113 between the first main surface 111 and the second main surface 112. The first main surface 111 is a surface on the front side of the stretchable device 1, and the second main surface 112 is a surface on the back side of the stretchable device 1, and is a surface to be attached to a living body. The main body portion 11 extends in a first direction A and is formed in a band shape. The peripheral edge 113 includes a first end edge 114 and a second end edge 115 extending along the first direction A and located on both sides in the second direction B orthogonal to the first direction A. The main body portion 11 has a first end portion 116 and a second end portion 117 located at both end portions in the first direction A. A width of the main body portion 11 is constant along the first direction A. The width refers to a length in the second direction B.

The projecting piece portion 15 is connected to the peripheral edge 113 so as to be bendable toward the second main surface 112. The projecting piece portion 15 includes a third main surface 151 continuous with the first main surface 111 and a fourth main surface 152 continuous with the second main surface 112 and facing the third main surface 151. That is, the third main surface 151 is flush with the first main surface 111, and the fourth main surface 152 is flush with the second main surface 112. The projecting piece portion 15 is formed integrally and continuously with the main body portion 11. A plurality of the projecting piece portions 15 are connected to the first end edge 114 and arranged side by side along the first direction A. The projecting piece portion 15 has a circular head portion and a neck portion narrower than the head portion, and the neck portion is connected to the main body portion 11. Note that the projecting piece portion 15 may be formed separately from the main body portion 11, and the projecting piece portion 15 may be attached to the main body portion 11 with an adhesive or the like.

An adhesive layer 16 is provided on the second main surface 112 of the main body portion 11 and the fourth main surface 152 of the projecting piece portion 15. The adhesive layer 16 includes, for example, an acrylic material, a urethane-based material, or a silicone-based material. As described above, by including the adhesive layer 16, when the projecting piece portion 15 is bent toward the second main surface 112, a state in which the projecting piece portion 15 is in contact with the second main surface 112 is easily maintained. Further, the stretchable device 1 can be attached to a living body by the adhesive layer 16. Note that the configuration may be such that the adhesive layer 16 is provided only on the second main surface 112. Hereinafter, in a diagram of the second to sixth embodiments, the adhesive layer 16 is omitted.

The bioelectrode 30 includes a conductive material having elasticity. As the conductive material, for example, a mixture of metal powder such as silver, copper, and the like and elastomer-based resin such as silicone is used. The bioelectrode 30 includes an electrode layer and a gel layer, and the gel layer preferably includes a conductive gel material containing water, a humectant, an electrolyte, and the like. By the above, adhesion to a living body is high, and a weak signal is easily detected.

The bioelectrode 30 is arranged on the third main surface 151. The bioelectrode 30 is formed by printing so as to be in direct contact with the third main surface 151, for example. Note that the bioelectrode 30 may be arranged on the third main surface 151 with another member such as an insulating member interposed between them. There are a plurality of the bioelectrodes 30, and each of the bioelectrodes 30 is arranged on each of the projecting piece portions 15.

The bioelectrode 30 is formed in a circular shape. A diameter of the bioelectrode 30 is preferably 5 mm to 40 mm, more preferably 10 mm to 30 mm, and still more preferably 10 mm to 20 mm. By the above, a biological signal can be efficiently measured. Note that a shape of the bioelectrode 30 may be an elliptical shape or a polygonal shape such as a quadrangular shape.

The stretchable wiring 20 includes a conductive material having elasticity. As the conductive material, for example, a mixture of metal powder such as silver, copper, and the like and elastomer-based resin such as silicone is used.

A large portion of the stretchable wiring 20 is arranged on the first main surface 111, and the other portion of the stretchable wiring 20 is arranged on the third main surface 151. That is, the stretchable wiring 20 includes a main-body-side wiring portion arranged on the first main surface 111 and a projecting piece side wiring portion arranged on the third main surface 151. The stretchable wiring 20 is formed by printing so as to be in direct contact with the first main surface 111, for example. Note that the stretchable wiring 20 may be arranged on the first main surface 111 with another member such as an insulating member interposed between them.

There are a plurality of the stretchable wirings 20, and each of the stretchable wirings 20 is connected to each of the bioelectrodes 30. Each of a plurality of the stretchable wirings 20 extends along the first direction A and is arranged side by side in the second direction B. Each of the stretchable wirings 20 extends from the first end portion 116 along the first direction A, and further extends in the second direction B toward a corresponding one of the bioelectrodes 30. Hereinafter, the second direction B is also referred to as a width direction of the main body portion 11.

In FIG. 1, the stretchable wiring 20 arranged closer to the first end edge 114 among a plurality of the stretchable wirings 20 is connected to the bioelectrode 30 arranged closer to the first end portion 116 among a plurality of the bioelectrodes 30. That is, a length in the first direction A of the stretchable wiring 20 increases as a position where the stretchable wiring 20 is arranged closer to the second end edge 115 from the first end edge 114.

A connection terminal 40 is connected to the first end portion 116. The connection terminal 40 is connectable to the second end portion 117, and the connection terminal 40 is attachable to and detachable from the second end portion 117. The connection terminal 40 is electrically connected to the stretchable wiring 20. The connection terminal 40 includes, for example, a connector and a control circuit. Note that a configuration in which the connection terminal 40 is attachable to and detachable from the second end portion 117 is not essential to the present invention.

Note that a plurality of the projecting piece portions 15 are connected to the first end edge 114, but may be connected to both the first end edge 114 and the second end edge 115.

Further, a plurality of the projecting piece portions 15 are connected to the first end edge 114, but the projecting piece portion 15 at a position farthest from the first end portion 116 may be connected to the second end portion 117. In other words, the projecting piece portion 15 is connected to the second end portion 117 located at an end portion in a direction parallel to the first direction A of the main body portion 11.

Further, the number of the projecting piece portions 15 is the same as the number of the bioelectrodes 30, but the number of the projecting piece portions 15 is not particularly limited. For example, a plurality of the bioelectrodes 30 may be arranged on one of the projecting piece portions 15. By reducing the number of the projecting piece portions, operation at the time of bending of the projecting piece portions can be simplified. Further, a shape of the projecting piece portion 15 is not limited to a circular shape, and may be, for example, a rectangular shape.

Further, a plurality of the stretchable wirings 20 may include a first group of stretchable wirings connected to the first end portion 116 and extending toward the second end portion 117, and a second group of stretchable wirings connected to the second end portion 117 and extending toward the first end portion 116. At this time, the first group of stretchable wirings are compared with each other, and a length in the first direction A of the stretchable wiring increases as a position where the stretchable wiring is arranged is closer to the second end edge 115 from the first end edge 114. Further, the second group of stretchable wirings are compared with each other, and a length in the first direction A of the stretchable wiring increases as a position where the stretchable wiring is arranged is closer to the second end edge 115 from the first end edge 114.

Next, a state of attachment preparation for attaching the stretchable device 1 to a living body will be described.

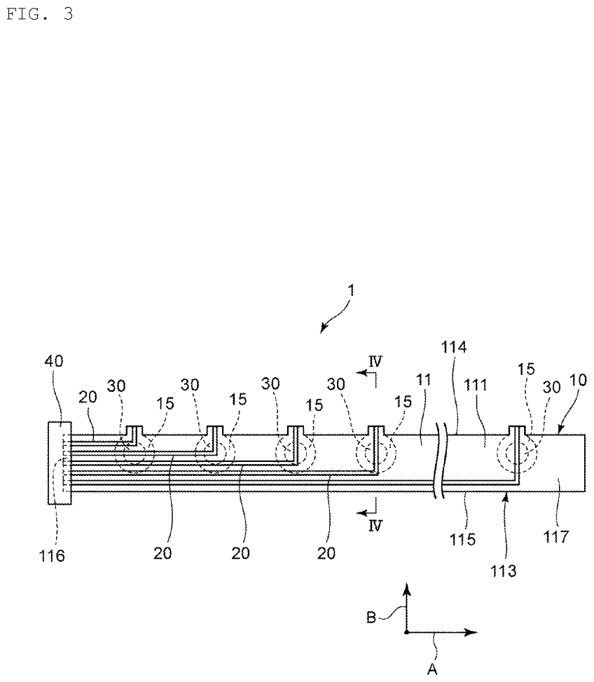

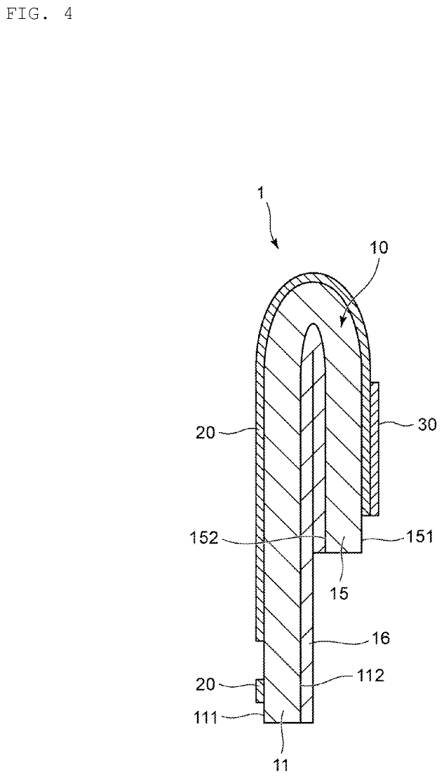

FIG. 3 is a plan view illustrating a preparation state for attachment of the stretchable device. FIG. 4 is a sectional view taken along IV-IV of FIG. 3. As illustrated in FIGS. 3 and 4, the projecting piece portion 15 is bent toward the second main surface 112 side from the state of FIGS. 1 and 2, the bioelectrode 30 is located on the second main surface 112 side with respect to the main body portion 11, and the stretchable device 1 is set in a state of attachment preparation.

At this time, the projecting piece portion 15 comes into contact with the adhesive layer 16. As described above, the projecting piece portion 15 being bent is brought into contact with the adhesive layer 16, so that the projecting piece portion 15 is fixed to the second main surface 112 of the main body portion 11 by the adhesive layer 16. By the above, positional deviation of the bioelectrode 30 with respect to a living body can be prevented, deviation of a signal detection position can be prevented, and detection accuracy can be enhanced. Further, the adhesive layer 16 plays a role of adhesion to a living body.

Further, the center of the bioelectrode 30 is arranged at a central portion in the width direction of the main body portion 11. The central portion in the width direction of the main body portion 11 refers to a central region when the first main surface 111 of the main body portion 11 is divided into three equal parts in the width direction. According to this, when the main body portion 11 is attached to a living body, the bioelectrode 30 can be easily installed at a desired position of the living body.

In the stretchable device 1 in the state of attachment preparation, when the stretchable device 1 is attached to a living body and a biological signal is detected, the second main surface 112 side of the main body portion 11 is attached to the living body, and the stretchable wiring 20 is located on the side opposite to the living body and hardly comes into contact with the living body.

On the other hand, the projecting piece portion 15 is bent toward second main surface 112 side, and the bioelectrode 30 is located on the second main surface 112 with respect to the main body portion 11. That is, the bioelectrode 30 is located on the side opposite to the stretchable wiring 20 with respect to the main body portion 11. For this reason, when the stretchable device 1 is attached to a living body and a biological signal is detected, a biological signal can be detected as the bioelectrode 30 is brought into contact with the living body.

Therefore, it is possible to reduce noise of a biological signal generated by the stretchable wiring 20 coming into contact with a living body, and it is possible to reduce discomfort of a subject generated by the stretchable wiring 20 coming into contact with skin.

Next, an attachment state in which the stretchable device 1 is attached to a human body will be described.

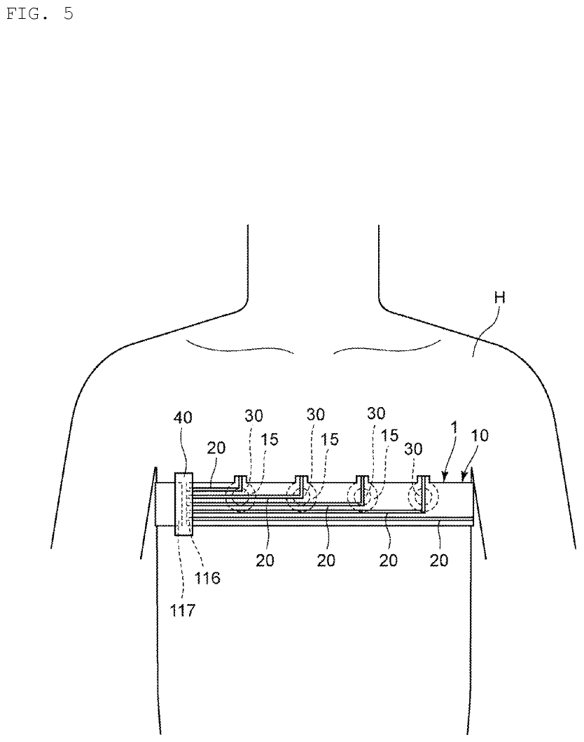

FIG. 5 is an explanatory diagram illustrating a use state of the stretchable device. As illustrated in FIG. 5, the stretchable device 1 in the attachment preparation state of FIG. 3 is attached to the chest of a human body H. Specifically, the main body portion 11 is wound around the human body H such that the bioelectrode 30 comes into contact with the human body H, and the second end portion 117 of the main body portion 11 is connected to the connection terminal 40. In this manner, the stretchable device 1 is fixed to the human body H, and a biological signal of the human body H is measured by the bioelectrode 30. Note that the stretchable device 1 may be wound around a portion other than the chest of the human body H.

As described above, while the bioelectrode 30 comes into contact with the human body H, the stretchable wiring 20 hardly comes into contact with the human body H, so that noise of a biological signal can be reduced, discomfort is not given to the human body H, and formation of a pressure point is also prevented. Further, since the stretchable device 1 can be fixed to the human body H by the connection terminal 40, it is possible to prevent deviation of a signal detection position. Further, since the main body portion 11 extends in the first direction A, the main body portion 11 can be wound around the human body H, and a biological signal can be detected in a wider range. As a result, detection accuracy can be enhanced. The stretchable device 1 is used for vital measurement such as an electrocardiogram (ECG), an electromyogram (EMG), an electroencephalogram (EEG), and electrical impedance tomography (EIT).

As described above, the stretchable device 1 is in a state in which the projecting piece portion 15 is not bent as illustrated in FIG. 1 before being used in an inspection, and is in a state in which the projecting piece portion 15 is bent as illustrated in FIG. 3 in a state of preparation for attachment to a living body. Therefore, the stretchable device 1 is in the state illustrated in FIG. 1 at the time of storage before use, and is in the state illustrated in FIG. 3 at the time of use.

### Second Embodiment

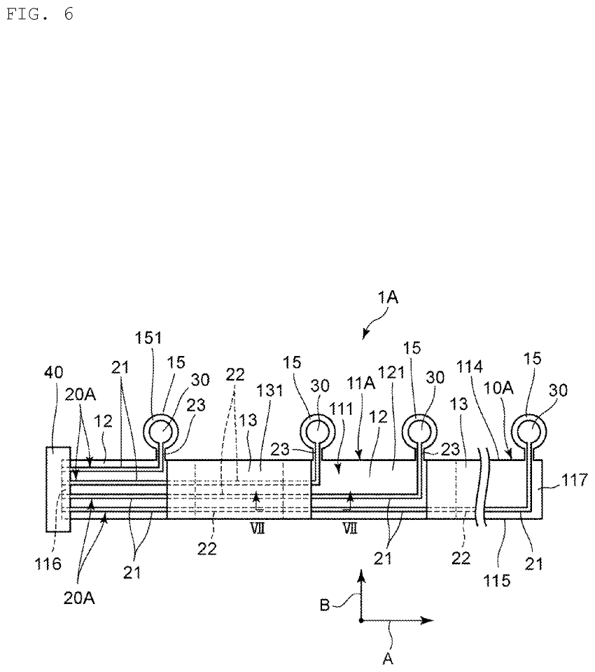

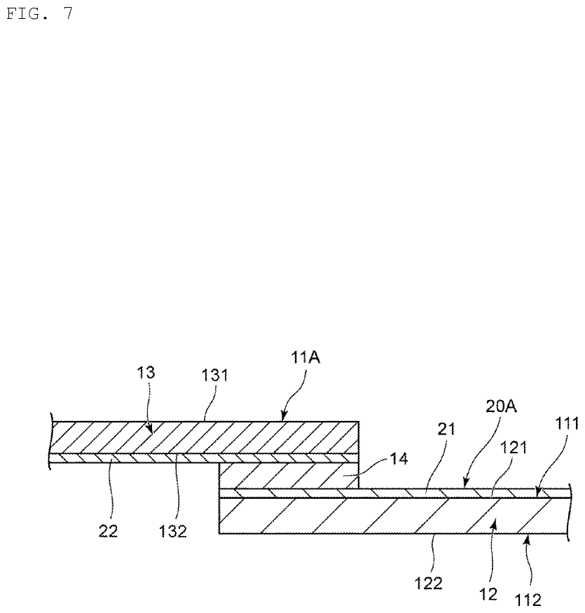

FIG. 6 is a plan view illustrating a second embodiment of the stretchable device. FIG. 7 is a sectional view taken along VII-VII of FIG. 6. The second embodiment is different from the first embodiment in the configuration of the stretchable substrate and the configuration of the stretchable wiring. This different configuration will be described below. The other configurations are the same as those of the first embodiment, denoted by the same reference symbols as those of the first embodiment, and omitted from description.

As illustrated in FIGS. 6 and 7, in a stretchable device 1A of the second embodiment, a main body portion 11A of a stretchable substrate 10A includes a plurality of individual piece portions 12 extending in the first direction A and arranged in the first direction A, and a coupling portion 13 connecting two of the individual piece portions 12 adjacent to each other in the first direction A.

The individual piece portion 12 includes a first main surface 121 and a second main surface 122 facing the first main surface 121. The coupling portion 13 includes a fifth main surface 131 and a sixth main surface 132 facing the fifth main surface 131. The individual piece portion 12 and the coupling portion 13 are arranged such that the first main surface 121 and the sixth main surface 132 face each other. Two of the individual piece portions 12 adjacent to each other in the first direction A are separated from each other. That is, a part of the coupling portion 13 is exposed from between two of the individual piece portions 12 adjacent to each other in the first direction A.

The first main surface 111 of the main body portion 11A includes the first main surface 121 of the individual piece portion 12, and the second main surface 112 of the main body portion 11A includes the second main surface 122 of the individual piece portion 12. Further, the main body portion 11A includes the fifth main surface 131 and the sixth main surface 132.

The projecting piece portion 15 is connected to the individual piece portion 12. The projecting piece portion 15 is formed integrally and continuously with the individual piece portion 12. The projecting piece portion 15 is not provided on the coupling portion 13. That is, the third main surface 151 is flush and continuous with the first main surface 121, and the fourth main surface 152 is flush and continuous with the second main surface 122.

A stretchable wiring 20A includes an individual piece side wiring portion 21 arranged on the first main surface 121, a coupling side wiring portion 22 arranged on the sixth main surface 132, and a projecting piece side wiring portion 23 arranged on the third main surface 151. That is, a part of the individual piece side wiring portion 21 is arranged on the first main surface 111, and a part of the coupling side wiring portion 22 is arranged on the second main surface 112. Note that, in FIG. 6, the stretchable wiring 20A closest to the first end edge 114 does not have the coupling side wiring portion 22.

The individual piece side wiring portion 21 and the projecting piece side wiring portion 23 are integrally and continuously connected. The individual piece side wiring portion 21 and the coupling side wiring portion 22 are electrically connected via a conductive member 14. The conductive member 14 is, for example, an anisotropic conductive film (ACF), and has conductivity in a thickness direction and insulation in a planar direction. The conductive member 14 contains thermosetting epoxy resin and a conductive particle contained in resin. Therefore, the individual piece side wiring portion 21 is conductive only to the coupling side wiring portion 22 immediately above the individual piece side wiring portion 21, and is not conductive to other ones of the individual piece side wiring portions 21 adjacent in a direction of the first main surface 121 or another one of the coupling side wiring portions 22 not immediately above.

The individual piece portion 12 and the coupling portion 13 are bonded by the conductive member 14. Note that, in a case where the conductive member 14 does not have adhesiveness, the individual piece portion 12 and the coupling portion 13 may be bonded by another bonding member.

A length in the first direction A of the individual piece portion 12 and a length in the first direction A of the coupling portion 13 are preferably the same. By the above, the individual piece portion 12 and the coupling portion 13 can have the same shape, and the individual piece portion 12 and the coupling portion 13 can be manufactured by the same process.

According to the above configuration, a length in the first direction A of the main body portion 11A can be adjusted by adjustment of the number of the individual piece portions 12. Further, the main body portion 11A can be manufactured by being divided into the individual piece portion 12 and the coupling portion 13, and the yield is improved.

Further, since two of the individual piece portions 12 adjacent to each other in the first direction A are separated from each other, if lengths of a plurality of the individual piece portions 12 are constant, a length in the first direction A of the main body portion 11A can be adjusted by adjustment of a length of the coupling portion 13. Further, lengths of a plurality of the individual piece portions 12 can be made constant, and a plurality of the individual piece portions 12 can be easily manufactured.

Note that the number of the individual piece portions 12 and the number of the coupling portions 13 are not particularly limited. Further, the number of the projecting piece portions 15 connected to one of the individual piece portions 12 is not particularly limited.

### Third Embodiment

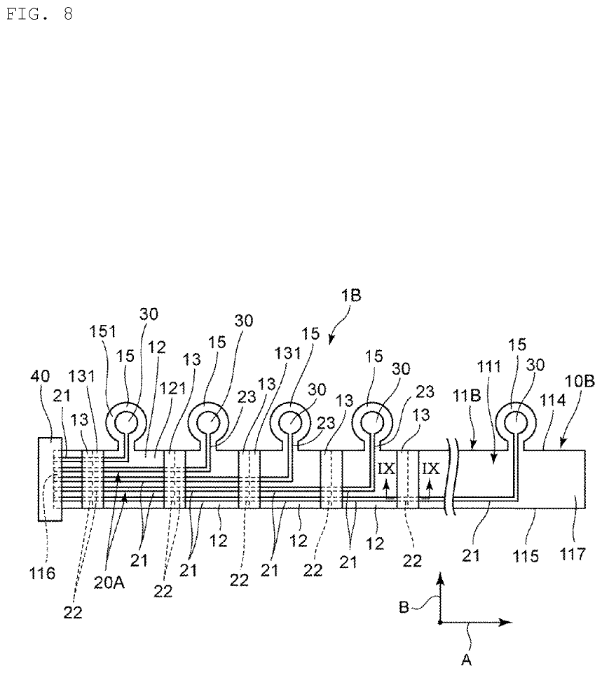

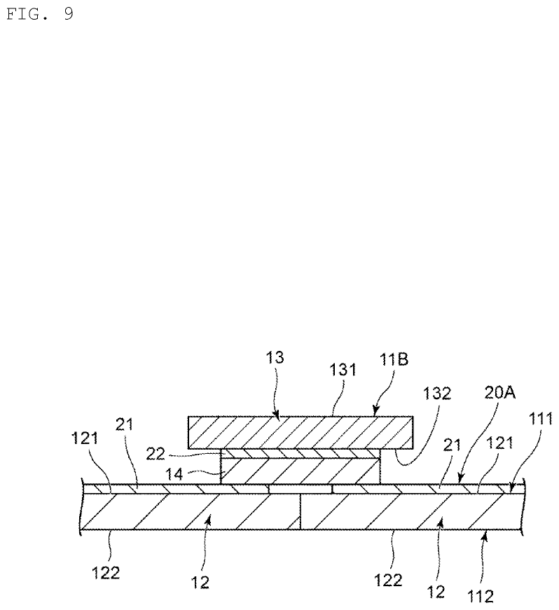

FIG. 8 is a plan view illustrating a third embodiment of the stretchable device. FIG. 9 is a sectional view taken along IX-IX of FIG. 8. The third embodiment is different from the second embodiment in the configuration of the stretchable substrate. This different configuration will be described below. The other configurations are the same as those of the second embodiment, and are denoted by the same reference symbols as those of the second embodiment, and omitted from description.

As illustrated in FIGS. 8 and 9, in a stretchable device 1B of the third embodiment, a main body portion 11B of the stretchable substrate 10B has a plurality of the individual piece portions 12 arranged in the first direction A and the coupling portion 13 that connects two of the individual piece portions 12 adjacent to each other in the first direction A. In the second embodiment, two of the individual piece portions 12 adjacent to each other in the first direction A are separated from each other, but in the third embodiment, two of the individual piece portions 12 adjacent to each other in the first direction A are in contact with each other. That is, a part of the coupling portion 13 is not exposed from between two of the individual piece portions 12 adjacent to each other in the first direction A.

Preferably, two of the individual piece portions 12 adjacent to each other in the first direction A are in overall contact with each other along the second direction B, but may be separated from each other in part. Since two of the individual piece portions 12 adjacent to each other are in overall contact with each other, it is possible to prevent exposure of the coupling side wiring portion 22 of the coupling portion 13 and to reduce noise.

A length in the first direction A of the coupling portion 13 is preferably shorter than a length in the first direction A of the individual piece portion 12. By the above, the coupling portion 13 can be made small, and manufacturing cost of the coupling portion 13 can be reduced. Other configurations are the same as those of the second embodiment, and omitted from description.

In the third embodiment, the first main surface 111 of the main body portion 11B includes the first main surface 121 of the individual piece portion 12, and the second main surface 112 of the main body portion 11B includes the second main surface 122 of the individual piece portion 12. Further, the main body portion 11B includes the fifth main surface 131 and the sixth main surface 132.

According to the above configuration, since the coupling side wiring portion 22 of the stretchable wiring 20 is less likely to be exposed to the second main surface 112 side with respect to the main body portion 11, discomfort of a subject can be further reduced, and noise of a biological signal can be further reduced. Since an effect of the other configurations are the same as that of the second embodiment, description of the effect will be omitted.

### Fourth Embodiment

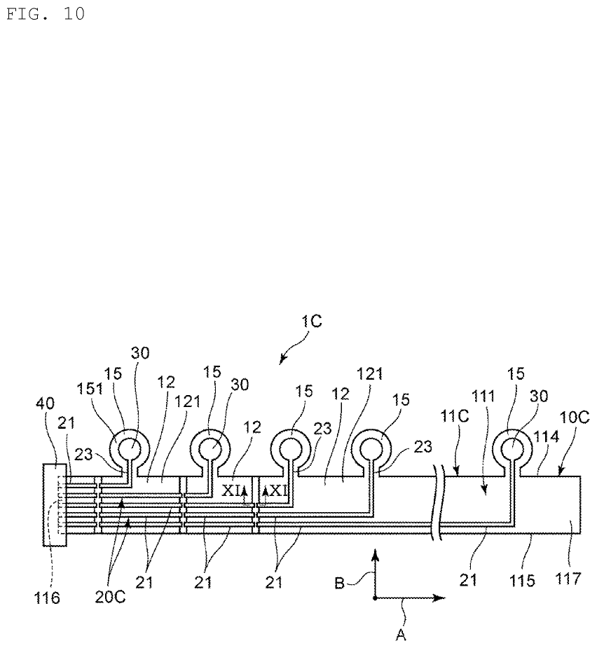

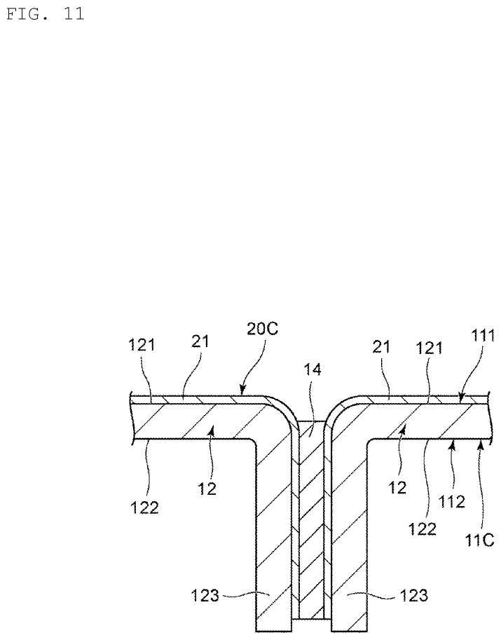

FIG. 10 is a plan view illustrating a fourth embodiment of the stretchable device. FIG. 11 is a sectional view taken along XI-XI of FIG. 10. The fourth embodiment is different from the second embodiment in the configuration of the stretchable substrate and the configuration of the stretchable wiring. This different configuration will be described below. The other configurations are the same as those of the second embodiment, and are denoted by the same reference symbols as those of the second embodiment, and omitted from description.

As illustrated in FIGS. 10 and 11, in a stretchable device 1C of the fourth embodiment, a main body portion 11C of a stretchable substrate 10C has a plurality of the individual piece portions 12 arranged in the first direction A. That is, the main body portion has the coupling portion in the second embodiment, but does not have the coupling portion in the fourth embodiment.

Further, in the stretchable device 1C, a stretchable wiring 20C includes the individual piece side wiring portion 21 arranged on the first main surface 121 and the projecting piece side wiring portion 23 arranged on the third main surface 151. That is, the stretchable wiring has the coupling side wiring portion in the second embodiment, but does not have the coupling side wiring portion in the fourth embodiment.

Then, in the above-described stretchable device 1C, in two of the individual piece portions 12 adjacent to each other in the first direction A, an end portion 123 of a first of the individual piece portions 12 on the side of a second of the individual piece portions 12 and the end portion 123 of the second of the individual piece portions 12 on the side of the first of the individual piece portions 12 are bent and connected such that the first main surface 121 of the end portion 123 of the first of the individual piece portions 12 and the first main surface 121 of the end portion 123 of the second of the individual piece portions 12 face each other. The end portion 123 of the first of the individual piece portions 12 and the end portion 123 of the second of the individual piece portions 12 are bonded by the conductive member 14.

The first main surface 111 of the main body portion 11C includes the first main surface 121 of the individual piece portion 12, and the second main surface 112 of the main body portion 11C includes the second main surface 122 of the individual piece portion 12.

The individual piece side wiring portion 21 arranged in the first of the individual piece portions 12 and the individual piece side wiring portion 21 arranged in the second of the individual piece portions 12 face each other at the end portion 123 of the first of the individual piece portions 12 and the end portion 123 of the second of the individual piece portions 12, and are electrically connected via the conductive member 14.

According to the above configuration, a length in the first direction A of the main body portion 11C can be adjusted by adjustment of the number of the individual piece portions 12. Further, the main body portion 11C can be manufactured by being divided into the individual piece portions 12, and the yield is improved. Further, since the stretchable wiring 20C is less likely to be exposed to the second main surface 112 side with respect to the main body portion 11C, discomfort of a subject can be further reduced, and noise of a biological signal can be further reduced. Since an effect of the other configurations are the same as that of the second embodiment, description of the effect will be omitted.

### Fifth Embodiment

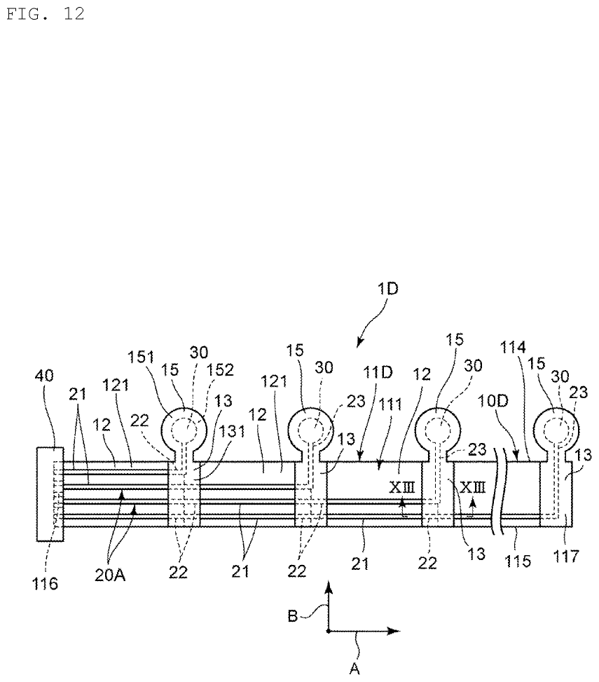

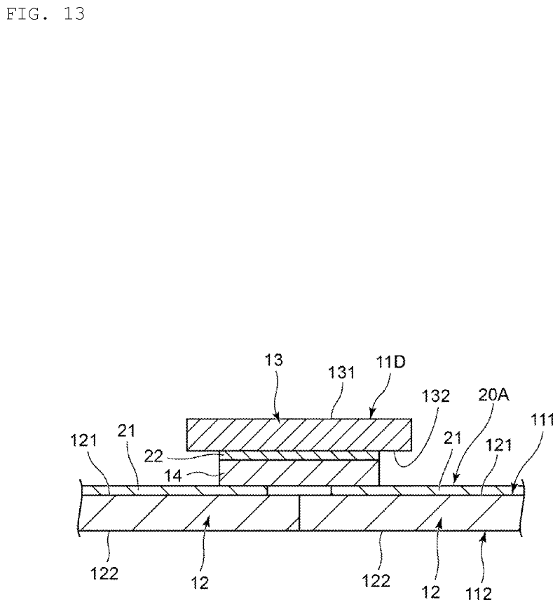

FIG. 12 is a plan view illustrating a fifth embodiment of the stretchable device. FIG. 13 is a sectional view taken along XIII-XIII of FIG. 12. The fifth embodiment is different from the third embodiment in the position of the bioelectrode and the configuration of the stretchable substrate. This different configuration will be described below. The other configurations are the same as those of the third embodiment, and are denoted by the same reference symbols as those of the third embodiment, and omitted from description.

As illustrated in FIGS. 12 and 13, in a stretchable device 1D of the fifth embodiment, the bioelectrode 30 is arranged on the fourth main surface 152 of the projecting piece portion 15. The projecting piece portion 15 is arranged with respect to the main body portion 11D such that the third main surface 151 is along the first main surface 111 and the fourth main surface 152 is along the second main surface 112. That is, the projecting piece portion 15 is not bent toward the first main surface 111 side and the second main surface 112 side. Specifically, the projecting piece portion 15 is arranged with respect to the main body portion 11D such that the third main surface 151 is parallel to the first main surface 111 and the fourth main surface 152 is parallel to the second main surface 112. Here, “parallel” includes not only being perfectly parallel, but also being substantially parallel to the extent that the projecting piece portion 15 is not bent toward the first main surface 111 side and the second main surface 112 side.

In the stretchable substrate 10D, the projecting piece portion 15 is connected to the coupling portion 13. The projecting piece portion 15 is formed integrally and continuously with the coupling portion 13. The projecting piece portion 15 is not provided on the individual piece portion 12. That is, the third main surface 151 is flush and continuous with the fifth main surface 131, and the fourth main surface 152 is flush and continuous with the sixth main surface 132.

The projecting piece side wiring portion 23 is arranged on the fourth main surface 152, and the bioelectrode 30 is connected to the projecting piece side wiring portion 23. The projecting piece side wiring portion 23 is connected to the coupling side wiring portion 22, and the coupling side wiring portion 22 is electrically connected to the individual piece side wiring portion 21 via the conductive member 14. By the above, the bioelectrode 30 is electrically connected to the individual piece side wiring portion 21.

According to the above configuration, the bioelectrode 30 is arranged on the fourth main surface 152, and the projecting piece portion 15 is arranged with respect to the main body portion 11D such that the third main surface 151 is parallel to the first main surface 111, and the fourth main surface 152 is parallel to the second main surface 112. For this reason, if the projecting piece portion 15 is not bent, the bioelectrode 30 exists on the side opposite to the stretchable wiring 20A with respect to the main body portion 11D, so that discomfort of a subject can be reduced and noise of a biological signal can be reduced. Further, since there is no need for bending, inspection can be performed quickly. Further, in a case where the stretchable device 1D is repeatedly used for inspection, deterioration due to bending of the projecting piece portion 15 hardly occurs. Since an effect of the other configurations are the same as that of the third embodiment, description of the effect will be omitted.

### Sixth Embodiment

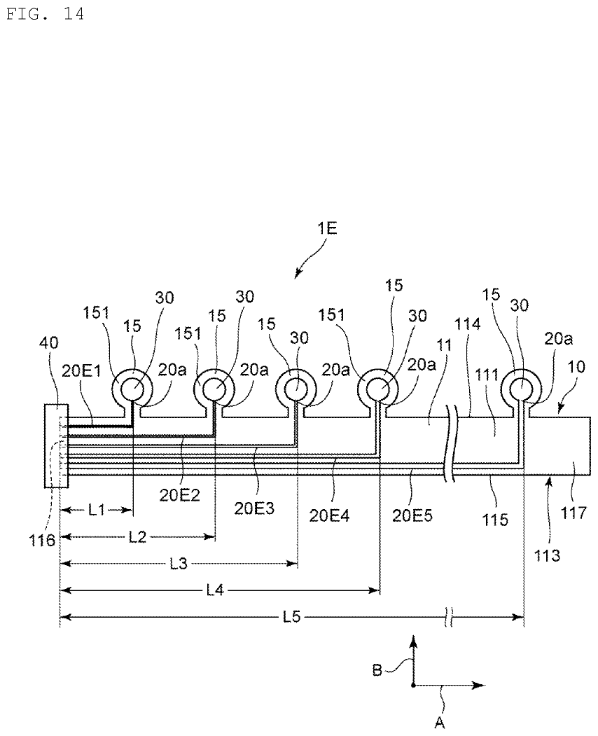

FIG. 14 is a plan view illustrating a sixth embodiment of the stretchable device. The sixth embodiment is different from the first embodiment in the configuration of the stretchable wiring. This different configuration will be described below. The other configurations are the same as those of the first embodiment, denoted by the same reference symbols as those of the first embodiment, and omitted from description.

As illustrated in FIG. 14, in a stretchable device 1E of the sixth embodiment, each of stretchable wirings 20E1 to 20E5 extends from the first end portion 116 along the first direction A, and each of the bioelectrodes 30 is electrically connected to each of the stretchable wirings 20E1 to 20E5. In each of the stretchable wirings 20E1 to 20E5, as distances L1 to L5 in the first direction A from the first end portion 116 to a portion 20a connected to the bioelectrode 30 increase, a width of the stretchable wirings 20E1 to 20E5 increases.

Specifically, the first stretchable wiring 20E1, the second stretchable wiring 20E2, the third stretchable wiring 20E3, the fourth stretchable wiring 20E4, and the fifth stretchable wiring 20E5 are arranged in parallel in order from the first end edge 114 toward the second end edge 115. The first distance L1 of the first stretchable wiring 20E1, the second distance L2 of the second stretchable wiring 20E2, the third distance L3 of the third stretchable wiring 20E3, the fourth distance L4 of the fourth stretchable wiring 20E4, and the fifth distance L5 of the fifth stretchable wiring 20E5 are longer in this order, and a width of the first stretchable wiring 20E1, a width of the second stretchable wiring 20E2, a width of the third stretchable wiring 20E3, a width of the fourth stretchable wiring 20E4, and a width of the fifth stretchable wiring 20E5 are larger in this order.

According to the above configuration, a biological signal is weak and is likely to attenuate while being transmitted from a detection position to a detector. By increasing a width of the stretchable wirings 20E1 to 20E5 according to a transmission distance (including the distances L1 to L5), signal attenuation hardly occurs in long-distance transmission, and a signal can be detected with high accuracy regardless of a signal transmission distance.

Note that, in a case where each of the stretchable wirings extends from one end portion of the first end portion and the second end portion along the first direction, in each of the stretchable wirings, a width of the stretchable wiring becomes wider as a distance in the first direction from the one end portion to a portion connected to the bioelectrode increases.

Further, a plurality of the stretchable wirings may include a first group of stretchable wirings connected to the first end portion and extending toward the second end portion, and a second group of stretchable wirings connected to the second end portion and extending toward the first end portion. At this time, the first group of stretchable wirings are compared with each other, and in each of the stretchable wirings, as a distance in the first direction from the first end portion to a portion connected to the bioelectrode increases, a width of the stretchable wiring increases. Further, the second group of stretchable wirings are compared with each other, and in each of the stretchable wirings, as a distance in the first direction from the second end portion to a portion connected to the bioelectrode increases, a width of the stretchable wiring increases.

Note that the present disclosure is not limited to the above-described embodiment, and can be modified in design without departing from the gist of the present disclosure. For example, feature points of the first to sixth embodiments may be combined in various ways.

In the above embodiment, the main body portion of the stretchable substrate has a band shape, but the shape is not limited to this shape. For example, the main body portion may have a non-long shape such as a polygon, a circle, or an ellipse. The projecting piece portion is connected to a peripheral edge of the main body portion, the stretchable wiring is provided on the main body portion, and the bioelectrode is provided on the projecting piece portion.

In the above embodiment, the main body portion of the stretchable substrate has a band shape and extends linearly, but may extend in a curved, meandering, or spiral shape.

In the above embodiment, the main body portion of the stretchable substrate has a band shape, and a width of the main body portion is constant along the first direction, but may change along the first direction. For example, the main body portion may have a rosary shape in which a plurality of circular portions are coupled, or may have a shape in which a circular portion and a band portion are alternately coupled.

In the above embodiment, a plurality of the projecting piece portions are connected to the first end edge. However, the configuration may be such that a plurality of the projecting piece portions are connected to at least one end edge of the first end edge and the second end edge. Although there are a plurality of the projecting piece portions, the number of the projecting piece portions may be one.

In the above embodiment, the projecting piece portion is connected to a peripheral edge of the main body portion, but may be connected to one of the first main surface and the second main surface of the main body portion, or may be connected to one of the first main surface and the second main surface together with the peripheral edge.

In the above embodiment, although there are a plurality of the stretchable wirings and a plurality of the bioelectrodes, the number of the stretchable wirings and the bioelectrodes may be one. In the above embodiment, all the stretchable wiring extends from the first end portion along the first direction, but each of the stretchable wirings may extend from one end portion of the first end portion and the second end portion along the first direction.

In the first embodiment, the stretchable wiring is arranged on the first main surface and the third main surface. However, as long as the bioelectrode is in contact with the first main surface, all the stretchable wirings may be arranged on the first main surface. Further, the configuration may be such that at least a part of the stretchable wirings is arranged on the first main surface, and the other part is arranged on the second main surface.

In the above embodiment, the stretchable wiring has the projecting piece side wiring portion, but the projecting piece side wiring portion does not need to be provided as long as the bioelectrode provided on the projecting piece portion is arranged so as to be close to an end edge (first main surface) of the main body portion.

### Seventh Embodiment

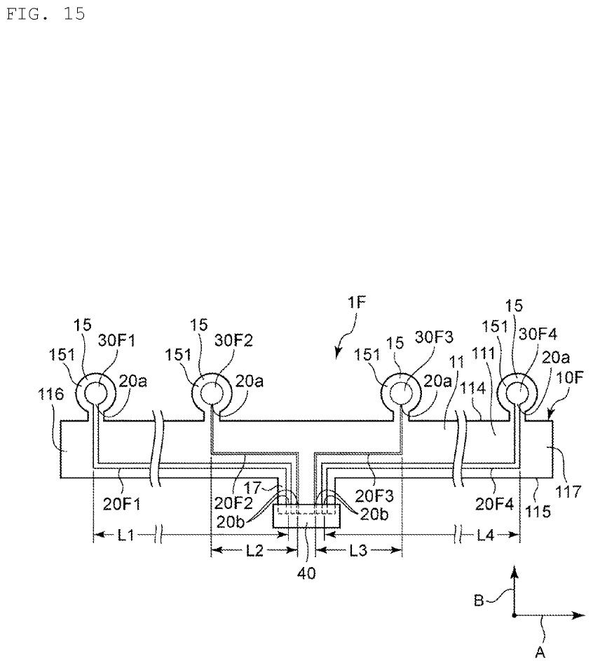

FIG. 15 is a plan view illustrating a seventh embodiment of the stretchable device. The seventh embodiment is different from the first embodiment in that a connection portion is further included, and in the configuration of the stretchable wiring. This different configuration will be described below. The other configurations are the same as those of the first embodiment, denoted by the same reference symbols as those of the first embodiment, and omitted from description.

As illustrated in FIG. 15, in a stretchable device 1F of the seventh embodiment, a stretchable substrate 10F further includes a connection portion 17. The connection portion 17 is connected to the second end edge 115 located at an end portion in the second direction B of the main body portion 11. An end portion of the stretchable wirings 20F1 to 20F4 is arranged in the connection portion 17. In this embodiment, the connection portion 17 is located at a central portion in the first direction A of the second end edge 115.

The connection terminal 40 is attached to the connection portion 17 and connected to an end portion of the stretchable wirings 20F1 to 20F4. According to this, for example, an external device (not illustrated) can be connected to the connection terminal 40 and signal data can be transmitted to the external device. Various types of data can be measured or various types of data can be detected by an external device.

Each of a plurality of the stretchable wirings 20F1 to 20F4 extends from the connection terminal 40 to a plurality of bioelectrodes 30F1 to 30F4. In a plurality of the stretchable wirings 20F1 to 20F4, as the shortest distances L1 to L4 in the first direction A from a portion 20b connected to the connection terminal 40 to the portion 20a connected to the bioelectrodes 30F1 to 30F4 are longer, a width of the stretchable wirings 20F1 to 20F4 becomes larger.

Specifically, the first bioelectrode 30F1, the second bioelectrode 30F2, the third bioelectrode 30F3, and the fourth bioelectrode 30F4 are arranged in parallel in order from the first end portion 116 toward the second end portion 117. That is, the second bioelectrode 30F2 and the third bioelectrode 30F3 are arranged closer to the connection portion 17 than the first bioelectrode 30F1 and the fourth bioelectrode 30F4. The first stretchable wiring 20F1 is connected to the first bioelectrode 30F1. The second stretchable wiring 20F2 is connected to the second bioelectrode 30F2. The third stretchable wiring 20F3 is connected to the third bioelectrode 30F3. The fourth stretchable wiring 20F4 is connected to the fourth bioelectrode 30F4.

In this embodiment, the first distance L1 of the first stretchable wiring 20F1 and the fourth distance L4 of the fourth stretchable wiring 20F4 are longer than the second distance L2 of the second stretchable wiring 20F2 and the third distance L3 of the third stretchable wiring 20F3. The first distance L1 and the fourth distance L4 are the same, and the second distance L2 and the third distance L3 are the same. Then, a width of the first stretchable wiring 20F1 and a width of the fourth stretchable wiring 20F4 are larger than a width of the second stretchable wiring 20F2 and a width of the third stretchable wiring 20F3. The width of the first stretchable wiring 20F1 and the width of the fourth stretchable wiring 20F4 are the same, and the width of the second stretchable wiring 20F2 and the width of the third stretchable wiring 20F3 are the same.

According to the above configuration, a biological signal is weak and is likely to attenuate while being transmitted from a detection position to a detector. By increasing a width of the stretchable wirings 20F1 to 20F4 according to a transmission distance (including the distances L1 to L4), signal attenuation hardly occurs in long-distance transmission, and a signal can be detected with high accuracy regardless of a signal transmission distance. Note that the first distance L1 to the fourth distance L4 may be different from each other, and as a length of the first distance L1 to the fourth distance L4 increases, a width of the stretchable wiring 20F1 to 20F4 corresponding to each distance increases.

In this embodiment, the stretchable device 1F may be attached to a human body directly, or may be brought into close contact with a human body by bringing the first end portion 116 and the second end portion 117 into close contact with each other and forming the stretchable device 1F into a belt shape. With such a structure, a position of the connection terminal 40 can be adjusted. As described above, the stretchable device 1F can measure various positions of a human body. Further, by adjusting a position of the connection terminal 40, the stretchable device 1F can be adjusted in an optimum shape according to a measurement position.

Further, the connection terminal 40 is not arranged at the first end portion 116 and the second end portion 117. That is, since it is also possible not to arrange the stretchable wiring at the first end portion 116 and the second end portion 117, the first end portion 116 and the second end portion 117 can be connected in various forms. Specifically, the first end portion 116 and the second end portion 117 may be connected so as to vertically overlap each other, or the first end portion 116 and the second end portion 117 may be integrated. As described above, various connection methods can be made possible, and the stretchable device 1F can be changed to an optimal structure according to a measurement position.

Further, a shape of the connection portion 17 is not particularly limited, and may be a rectangle as illustrated in FIG. 15 or may be a circle. Further, a position in the first direction A of the connection portion 17 is not particularly limited, but is preferably a central portion in the first direction A. This makes it possible to arrange the stretchable wiring and the bioelectrode symmetrically with respect to the central portion, so that printing of the stretchable wiring and the bioelectrode is facilitated. Further, the connection portion 17 may be connected to the first end edge 114.

Note that the stretchable device 1F may include a plurality of the connection terminals 40, each of which may be arranged at any of the first end portion 116, the second end portion 117, and the connection portion 40. By connecting to the connection terminal 40 closest to each of the bioelectrodes 30, a length of the stretchable wiring 20 can be reduced in a case where the stretchable device 1F is sufficiently long in the first direction.

Note that each of the embodiments is an example, and the present invention is not limited to each of the embodiments. Further, partial replacement or combination of configurations shown in different embodiments is possible.

### DESCRIPTION OF REFERENCE SYMBOLS

1, 1A to 1F: Stretchable device
10, 10A to 10D, 10F: Stretchable substrate
11, 11A to 11D: Main body portion
111: First main surface
112: Second main surface
113: Peripheral edge
114: First end edge
115: Second end edge
116: First end portion
117: Second end portion
12: Individual piece portion
121: First main surface
122: Second main surface
123: End portion
13: Coupling portion
131: Fifth main surface
132: Sixth main surface
14: Conductive member
15: Projecting piece portion
151: Third main surface
152: Fourth main surface
16: Adhesive layer
17: Connection portion
20, 20A, 20C: Stretchable wiring
20a: Portion connected to bioelectrode of stretchable wiring
20b: Portion connected to connection terminal of stretchable wiring
20E1 to 20E5: First to fifth stretchable wirings
20F1 to 20F4: First to fourth stretchable wirings
21: Individual piece side wiring portion
22: Coupling side wiring portion
23: Projecting piece side wiring portion
30: Bioelectrode
30F1 to 30F4: First to fourth bioelectrodes
40: Connection terminal
A: First direction
B: Second direction
L1 to L5: First to fifth distances

1. A stretchable device comprising:
a stretchable substrate that includes:
a main body portion having a first main surface and a second main surface facing the first main surface; and
a projecting piece portion connected to the main body portion, the projecting piece portion having a third main surface continuous with the first main surface and a fourth main surface continuous with the second main surface and facing the third main surface;
a stretchable wiring, wherein at least a part of the stretchable wiring is arranged on the first main surface of the main body portion of the stretchable substrate; and
a bioelectrode arranged on the third main surface of the projecting piece portion of the stretchable substrate and electrically connected to the stretchable wiring.

2. The stretchable device according to claim 1, wherein
the main body portion extends in a first direction, and
the projecting piece portion is connected to an end edge of the main body portion located at an end portion in a second direction orthogonal to the first direction of the main body portion.

3. The stretchable device according to claim 1, wherein the projecting piece portion is bendable toward a side of the second main surface.

4. The stretchable device according to claim 1, wherein the projecting piece portion is in a bent position toward a side of the second main surface.

5. The stretchable device according to claim 4, wherein
the main body portion extends in a first direction, and
a center of the bioelectrode is arranged at a central portion in a second direction orthogonal to the first direction of the main body portion.

6. The stretchable device according to claim 3, further comprising:
an adhesive layer on the second main surface.

7. The stretchable device according to claim 4, further comprising:
an adhesive layer on the second main surface, wherein
the projecting piece portion is fixed to the second main surface by the adhesive layer.

8. The stretchable device according to claim 1, wherein
the main body portion extends in a first direction, and includes:
a plurality of individual piece portions that include the first main surface and the second main surface and are arranged in the first direction; and
a coupling portion that includes a fifth main surface and a sixth main surface facing the fifth main surface and connects two of the individual piece portions adjacent to each other in the first direction,
each of the individual piece portions and the coupling portion are arranged such that the first main surface and the sixth main surface face each other,
the projecting piece portion is connected to the individual piece portion,
the stretchable wiring includes an individual piece side wiring portion arranged on the first main surface and a coupling side wiring portion arranged on the sixth main surface, and
the individual piece side wiring portion and the coupling side wiring portion are electrically connected.

9. The stretchable device according to claim 1, wherein
the main body portion extends in a first direction and includes a plurality of individual piece portions including the first main surface and the second main surface and arranged in the first direction,
the projecting piece portion is connected to each of the individual piece portions,
the stretchable wiring includes an individual piece side wiring portion arranged on the first main surface,
between two of the individual piece portions adjacent to each other in the first direction, an end portion of a first of the individual piece portions on a side of a second of the individual piece portions and an end portion of the second of the individual piece portions on a side of the first of the individual piece portions are bent and coupled such that a first main surface of the end portion of the first of the individual piece portions and a first main surface of the end portion of the second of the individual piece portions face each other, and
the individual piece side wiring portion arranged in the first of the individual piece portions and the individual piece side wiring portion arranged in the second of the individual piece portions are electrically connected.

10. The stretchable device according to claim 1, wherein
the main body portion extends in a first direction and has a first end portion and a second end portion on both sides in the first direction, the stretchable device further comprising:
a connection terminal connected to the first end portion and connectable to the second end portion.

11. The stretchable device according to claim 1, wherein the stretchable device includes:
a plurality of the stretchable wirings;
a plurality of the projecting piece portions; and
a plurality of the bioelectrodes,
the main body portion extends in a first direction and has a first end portion and a second end portion on both sides thereof in the first direction,
each of the plurality of the stretchable wirings extends along the first direction from one end portion of the first end portion and the second end portion,
each of the plurality of the bioelectrodes is electrically connected to the plurality of the stretchable wirings, and
in the plurality of the stretchable wirings, as a distance in the first direction from the one end portion to a portion connected to the bioelectrode increases, a width of the stretchable wiring increases.

12. The stretchable device according to claim 1, wherein
the main body portion extends in a first direction, and
the stretchable substrate further includes a connection portion connected to an end edge located at an end portion in a second direction orthogonal to the first direction in the main body portion and in which an end portion of the stretchable wiring is arranged, the stretchable device further comprising:
a connection terminal attached to the connection portion and connected to the end portion of the stretchable wiring.

13. The stretchable device according to claim 12, wherein the stretchable device includes:
a plurality of the stretchable wirings;
a plurality of the projecting piece portions; and
a plurality of the bioelectrodes,
each of the plurality of the stretchable wirings extends from the connection terminal to the plurality of the bioelectrodes, and
in the plurality of the stretchable wirings, as a distance in the first direction from a portion connected to the connection terminal to a portion connected to the bioelectrode increases, a width of the stretchable wiring increases.

14. A stretchable device comprising:
a stretchable substrate that includes:
a main body portion having a first main surface and a second main surface facing the first main surface; and
a projecting piece portion connected to the main body portion, the projecting piece portion having a third main surface and a fourth main surface facing the third main surface, and the projecting piece portion being arranged with respect to the main body portion such that the third main surface is along the first main surface and the fourth main surface is along the second main surface;
a stretchable wiring, wherein at least a part of the stretchable wiring is arranged on the first main surface of the main body portion of the stretchable substrate; and
a bioelectrode arranged on the third main surface of the projecting piece portion of the stretchable substrate and electrically connected to the stretchable wiring.

15. The stretchable device according to claim 14, wherein
the main body portion extends in a first direction, and includes:
a plurality of individual piece portions that include the first main surface and the second main surface and are arranged in the first direction; and
a coupling portion that includes a fifth main surface and a sixth main surface facing the fifth main surface and connects two of the individual piece portions adjacent to each other in the first direction,
each of the individual piece portions and the coupling portion are arranged such that the first main surface and the sixth main surface face each other,
the projecting piece portion is connected to the coupling portion,
the stretchable wiring includes an individual piece side wiring portion arranged on the first main surface and a coupling side wiring portion arranged on the sixth main surface, and
the individual piece side wiring portion and the coupling side wiring portion are electrically connected.

16. The stretchable device according to claim 14, wherein
the main body portion extends in a first direction and has a first end portion and a second end portion on both sides thereof in the first direction, the stretchable device further comprising:
a connection terminal connected to the first end portion and connectable to the second end portion.

17. The stretchable device according to claim 14, wherein the stretchable device includes:
a plurality of the stretchable wirings;
a plurality of the projecting piece portions; and
a plurality of the bioelectrodes,
the main body portion extends in a first direction and has a first end portion and a second end portion on both sides thereof in the first direction,
each of the plurality of the stretchable wirings extends along the first direction from one end portion of the first end portion and the second end portion,
each of the plurality of the bioelectrodes is electrically connected to the plurality of the stretchable wirings, and
in the plurality of the stretchable wirings, as a distance in the first direction from the one end portion to a portion connected to the bioelectrode increases, a width of the stretchable wiring increases.

18. The stretchable device according to claim 14, wherein
the main body portion extends in a first direction, and
the stretchable substrate further includes a connection portion connected to an end edge located at an end portion in a second direction orthogonal to the first direction in the main body portion and in which an end portion of the stretchable wiring is arranged, the stretchable device further comprising:
a connection terminal attached to the connection portion and connected to the end portion of the stretchable wiring.

19. The stretchable device according to claim 18, wherein the stretchable device includes:
a plurality of the stretchable wirings;
a plurality of the projecting piece portions; and
a plurality of the bioelectrodes,
each of the plurality of the stretchable wirings extends from the connection terminal to the plurality of the bioelectrodes, and
in the plurality of the stretchable wirings, as a distance in the first direction from a portion connected to the connection terminal to a portion connected to the bioelectrode increases, a width of the stretchable wiring increases.
